(19) United States
(12) Patent Application Publication
(10) Pub. No.: US 2026/0210952 A1
(43) Pub. Date: Jul. 23, 2026

(54) METHOD AND SYSTEM FOR FAST DIAGNOSIS OF CANCER AND OTHER DISEASES IN TISSUE SAMPLES

(71) Applicant: Scorpido Photonics Inc., San Pedro, CA (US)

(72) Inventor: Dmitri Lapotko, San Pedro, CA (US)

(21) Appl. No.: 19/140,937

(22) PCT Filed: Dec. 19, 2023
(86) PCT No.: PCT/US23/84719

§ 371 Date: Jun. 18, 2025

Related U.S. Application Data
(60) Provisional application No. 63/434,556, filed on Dec. 22, 2022.

(51) Int. Cl. G01N 33/543

(52) U.S. Cl. CPC G01N 33/54346; G01N 33/54373

(57) ABSTRACT

The invention relates to all-optical methods and systems for diagnosis of disease (for example, cancer) with the cell-level precision and in real time in ex Vivo tissue samples (biopsies or surgical specimens) through the generation and detection of disease-specific plasmonic nanobubbles (“PNBs”). PNBs are on-demand short laser pulse-activated non-stationary vapor nanobubbles. A system for diagnosing the disease comprises a device which optically generates and detects PNBs in a sample. A method comprises (a) incubating a sample with plasmonic nanoparticles; (b) placing a sample into the PNB system: (c) generating PNBs with a pump laser pulse; (d) detecting PNBs optically in a sample; and (e) diagnosing the disease through analysis of the detected optical signal in response to a laser pulse. PNB system and method provide a rapid micro-analysis of ex viva samples, solid or liquid, with a single cell sensitivity.

### BACKGROUND

The present invention relates to methods and systems for diagnosis of tissue samples ex vivo through plasmonic nanobubbles (“PNBs”).

Analysis of tissue samples, liquid, soft or hard tissue, is an integral part of two major fields in medicine: surgery and primary diagnostics/screening. Currently, both rely on biopsy and histopathology, a lengthy procedure which often hinges on a qualification of the pathologist who examines samples under the microscope. Both fields therefore suffer from low diagnostic speed, with limited ability to use the diagnostic data for treatment during the diagnostic or surgical procedure. However, it is a robotic diagnostics and surgery, with their ability to deliver precise treatment, that experience major setbacks from the lack of rapid diagnostic data. Such data would guide a manual or robotic treatment during the main (diagnostic) procedure, thus enabling robotic medicine to achieve a performance well beyond what human doctors can do. For example, surgery often remains blind for a microscopic residual disease (MRD) in a surgical margin: MRD often cannot be detected and treated intraoperatively, especially if it is represented by low number of residual cancer cells, unresectable or undetectable with current means. This results in excessive morbidity, additional unsafe radiation/chemo therapies, tumor recurrence and metastases, with poor clinical outcome and quality of life, and also increases costs of the treatment.

In robotic surgery, an intraoperative real-time diagnostic data on cancer status of surgical margins at the cell level would allow to precisely detect the presence of MRD (residual cancer cells), a major source of poor clinical outcomes in surgery of aggressive and often unresectable tumors. Such intraoperative data can be used by surgical robot to precisely localize and excise an area with residual cancer cells, thus reducing a morbidity and improving a clinical outcome and quality of life. An express or a real-time ex vivo diagnostic option would be a “missing link” to turn many current robotic and manual surgical products into an efficient treatment well beyond standard surgery

In flexible diagnostic robotics, a cancer-positive real-time ex vivo data obtained during endoluminal biopsy procedure would allow delivery of treatment to the cancer-positive location through the same endoluminal device and within the same procedure. In case of aggressive and quickly growing cancers, such as a lung cancer, for example, such acceleration of cancer diagnosis to a real-time and improvement of diagnostic precision to the level of single cancer cells would treat the disease in its earliest, with much better chances for a cure. With several robotic flexible and surgical products available in clinic, for example, endoluminal diagnostic robotics, the express diagnostic option would be a “missing link” to turn these new products into an efficient treatment well beyond standard diagnostics and screening.

PNBs are non-stationary vapor nano- or micro-bubbles generated due to heat produced by plasmonic or light-absorbing nanoparticles or their clusters upon absorption and conversion of pulsed optical energy into a localized heat. Laser pulse-activated PNBs, with their optical and mechanical impact at nanoscale, can detect single cancer cells, precisely remove them from healthy tissue at micro-scale, and/or selectively destroy cancer cells without damaging healthy cells. See Nature Nanotech 2016 11, 525-532 https://doi.org/i0.1038/nnano.2015.343; Nature Med 2014 July; 20(7): 778-784 doi: 10.1038/nm.3484; J Surg Res 2011 March; 166(1): e3-e13 doi: 10.1016/j.jss.2010.10.039.

However, in previous disclosures of PNBs, administration of plasmonic nanoparticles or optical external energy or both were performed in vitro or directly in a patient (in vivo), not ex vivo. Real tissues present hurdles that are not seen ex vivo, for example, optoacoustic background in real tissues can interfere with the detection of PNBs. Detailed analysis of prior art for ex vivo application of PNB is in the table below:

| | Prior ex-vivo and in vitro PNB | |
|---|---|---|
| | diagnostics | This invention |
| Administration | In vivo, a long time, possible | Ex vivo, a short time, no safety nor |
| of gold | safety and regulatory concerns | regulatory restrictions |
| nanoparticles | | |
| Generation of | Ex vivo, free space pump beam of | Ex vivo, free space pump beam of small |
| PNBs | large diameter, >0.5 mm | diameter, down to 20 um, to improve |
| | | the a diagnostic sensitivity to a single |
| | | cancer cell, a small volume of tissue is |
| | | probed, to reduce the background |
| | | signal, and thus to improve the |
| | | detection sensitivity |
| Detection of | Ex vivo, acoustic signal from | Problem resolved: a high PNB detection |
| PNBs | PNBs, includes a background | sensitivity in tissue, with a new |
| | signal of optoacoustic origin from | combination of the method AND |
| | a large volume of tissue exposed | design: |
| | to a large pump laser beam | Method: Ex vivo, optical, |
| | Problem: poor PNB detection | Optical back-scattering by PNBs |
| | sensitivity in tissue | delivers a PNB-specific time-resolved |
| | | image or time-trace signal of non- |
| | | stationary nature in a specific time |
| | | domain, while the optical background |
| | | signal from a tissue has a stationary |
| | | nature. This difference in PNB and |
| | | background optical scattering signals |
| | | supports a high sensitivity optical |
| | | detection of PNB signal, such |
| | | sensitivity not available through |
| | | acoustic detection of same PNBs due to |
| | | much higher optoacoustic background |
| | | signal which is similar to PNB signal |
| | | Design: a small diameter optical probe |
| | | beam, combined with a very low |
| | | intensity noise of the probe light source, |
| | | allows to detect very small non- |
| | | stationary PNB-induced optical |
| | | scattering signals as deviations from a |
| | | stationary background by the tissue |
| | | surrounding PNBs, from a small area of |
| | | the tissue being exposed to a pump laser |
| | | beam, with the diameter of the probed |
| | | tissue area limited to 10-300 um |
| Optical | Optical detection of PNBs in tissue | Optical detection of PNBs in tissue uses |
| detection of | used a microscope with a probe | the back-scattered by PNB light which is |
| PNBs in tissue, | beam through the sample, and the | being detected on the same side of the |
| prior art (our | probe beam was detected on an | sample as the sample is exposed to the |
| publications | opposite side of the sample. The | pump and probe laser beams. This |
| Biomaterials | sample has to be optically | eliminates the requirements of the |
| 2010, | transparent for a probe laser beam | sample to be optically transparent for a |
| Nanotechnology | otherwise no signal can be detected | probe laser beam |
| 2010) | | |
| Detection of | A single cancer cell detection was | The highest diagnostic sensitivity up to a |
| target (cancer) | challenging due to a high | single target cell due to optical PNB |
| cells | background | detection method and a novel micro- |
| | | optical design |

Therefore, there is a need for fast ex vivo high sensitivity diagnosis of tissue samples. The present invention meets that need by providing for methods and systems for ex vivo diagnosis of tissue samples through optical generation and detection of PNBs, with ex vivo treatment of a sample (a hard, soft or a liquid) with plasmonic nanoparticles and optical energy.

### BRIEF DESCRIPTION OF THE DRAWINGS

FIG. 1 depicts the optical requirements for the delivery and collection of light to the tissue surface for ex vivo PNB diagnostics: 1—tissue sample mounted on an optically transparent surface, 2—PNB in a tissue surface, 3—the pump laser beam (solid) has the diameter D at the tissue surface, 10-400 um range, 4—probe laser beam (dashed) is co-delivered with the pump laser beam, 5—backscattered by PNB probe laser light propagates in all directions, with the focal point at the tissue surface 6, the PNB can be located within the depth h from the tissue surface, in the range 0-200 um.

FIG. 2 is a flow chart depicting a method and procedure comprising a combination of five connected components, each necessary for the PNB diagnostics of cancer ex vivo: (1) a short-term incubation of the sample with plasmonic nanoparticles, (2) mounting the sample onto optically-transparent holder, (3) generation of PNBs with a pump laser pulse, (4) detection of PNBs with a probe laser beam, (5) diagnosing the sample based upon the signal of the probe laser beam.

FIG. 3 depicts sample 1, PNB 2, pump laser beam 3 (solid) and probe laser beam 4 (dashed) are controlled with a low-NA lens 5 which provides a broad focal point in the sample 1 for the beams 3 and 4; beams 3 and 4 are delivered to the sample through the two holes in the mirror 7 and in the lens 6; back-scattered by a PNB probe laser light is collected by a high-NA lens 6 which collimates the back-scattered by a PNB probe laser light, the mirror 7 separates the backscattered light from the beams 3 and 4, the lens 8 collects the back-scattered light into a photodetector 9.

FIG. 4 depicts sample 1, PNB 2, pump laser beam 3 (solid) and probe laser beam 4 (dashed) are controlled with a low-NA lens 5 which provides a broad focal point in the sample 1 for the beams 3 and 4; beams 3 and 4 are delivered to the sample a compact axial mirror 7 and the hole in the lens 6; back-scattered by a PNB probe laser light is collected by a high-NA lens 6 which collimates the back-scattered by a PNB light, the mirror 7 has anti-reflective coating to transmit most of probe laser light through it, to be focused by the lens 8 into a photodetector 9.

FIG. 5 depicts sample 1, PNB 2, pump laser beam 3 (solid) and probe laser beam 4 (dashed) are delivered to the sample through an optical fiber 11 with its output distal tip positioned in the air before the light-collecting lens 6; the lens 6 concentrates the output pump laser beam into the sample to form the desired beam diameter in the sample; back-scattered by a PNB probe laser light is collected by a high-NA lens 6 which collimates the back-scattered by a PNB light, to direct it to the photodetector as shown in FIG. 4.

FIG. 6 depicts sample 1, PNB 2, pump laser beam 3 (solid) and probe laser beam 4 (dashed) are delivered to the sample with optical fiber system which includes an optical fiber 11, a coupler 7 (to launch the pump and probe laser beam into the optical fiber 11), the ferrule 10 (to mount the output distal tip of the fiber in the hole (shaft) of the light-collecting lens 6), and a micro-lens 5 that forms the desired diameter of the output pump laser beam in the sample 1, for example, 30 um; back-scattered by a PNB probe laser light is collected by a high-NA lens 6 which collimates the back-scattered by a PNB light, which is focused by the lens 8 into a photodetector 9.

FIG. 7 depicts an optically separated delivery and collection of light to/from the tissue sample 1 for optical generation and detection of a PNB 2: the pump 3 (solid) and probe 4 (dashed) laser beams are directed and concentrated onto the sample surface with the lens 5, the PNB-scattered probe laser light is collected with the lens 6 positioned at the angle A to the tissue surface.

FIG. 8A depicts a sample is exposed to collinear pump (solid) and probe (dashed) laser beams, FIG. 8B depicts exposure of a sample to a probe beam under total internal reflection conditions which occur in case of a PNB generation. 1—a pulsed pump laser, 2—a c.w. or pulsed probe laser, 3—a pump laser pulse (solid line), of specific diameter and fluence, to the sample, 4—a probe laser beam (dashed line), coincides with the pump beam at the sample surface and is co-delivered using same optics as for the pump laser beam, 5—an optically transparent glass sample holder with entrance and exit optical windows for a probe laser beam designed to provide an angle of incidence of a probe laser beam with a sample-holder surface which results in total internal reflection only in case the media on a sample side becomes a vapor, 6—a tissue sample, 7—a PNB generated around plasmonic nanoparticles in response to a pump laser pulse, 8—backscattered by a PNB probe laser light, 9—optical filter to pass only a probe laser light, 10—a photodetector, to detect the backscattered probe laser light, as a time-response or as a time-resolved image.

### DETAILED DESCRIPTION OF THE INVENTION

The present invention is directed to an all-optical method and system for ex vivo (i.e., a sample extracted from a patient and having viable tissues and cells) rapid micro-analysis of biopsy or surgical samples with a single target cell sensitivity, achieved through pump-probe laser probing a small volume sample and ex vivo processing of the tissue with plasmonic nanoparticles and laser radiation.

The present invention provides for diagnosis of tissue samples ex vivo (e.g., biopsies of soft, hard or liquid tissues, or surgical specimens) with PNBs, allowing for (i) shortened time to diagnosis, 10-30 min instead of days or weeks; (ii) non-invasive diagnosis, with no plasmonic nanoparticles or laser energy going into a patient; (iii) improved precision of the diagnostic process, compared to that in vivo, through a precise administration of gold nanoparticles and laser radiation to generate and detect PNBs in the ex vivo samples; (iv) improved clinical outcome by enabling PNB data-driven therapeutic response while having a biopsy tool in patient and during the biopsy procedure; and/or (v) improved clinical outcome through precise PNB data-driven complementary minimally invasive treatment of the target (a lung nodule or an MRD in a surgical bed) during the diagnostic or surgical procedure.

In a preferred embodiment, a tissue sample is incubated with gold nanoparticles and a laser device is utilized for detecting PNB ex vivo, the laser device capable of optical detection, free space- or fiber-supplied pump and probe laser beams with a picosecond (“ps”) pump laser pulse and a pulsed or continuous probe laser radiation.

A tissue sample may include hard, soft and liquid tissue, obtained through a biopsy or other diagnostic or medical procedure.

To achieve a high sensitivity and specificity of PNB diagnostics in ex vivo tissue samples several optical design conditions must be met as detailed below.

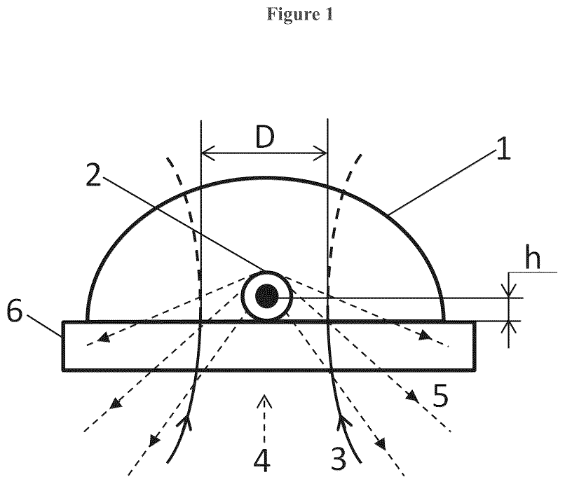

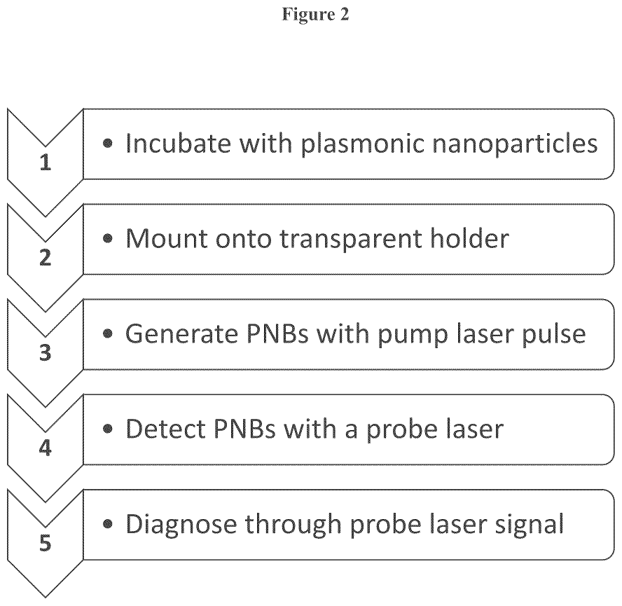

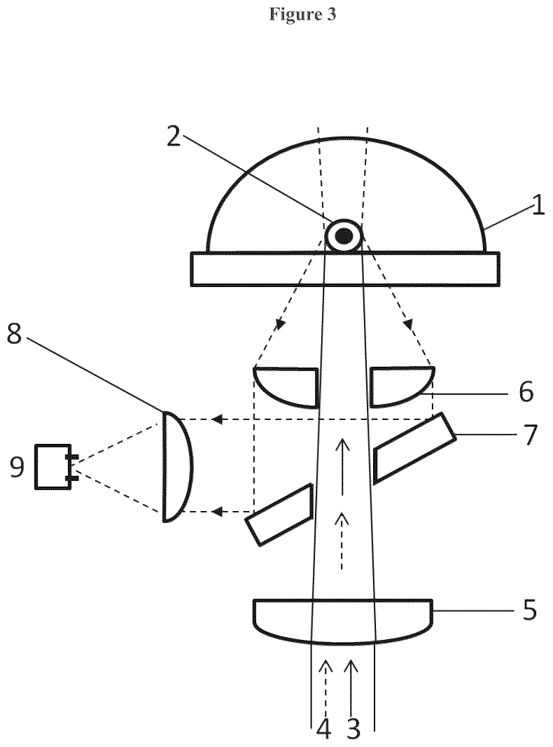

With reference to FIG. 1, efficient optical generation and detection of PNBs in a tissue surface layer requires the pump pulsed beam and the probe beam to be delivered to the tissue sample surface under specific diameter D, in the range 10 um-400 um, with uniform exposure of the tissue to both pump and probe laser beams. In technical terms, this requires delivering the collimated or focused two coinciding or coaxial laser beams from the pump and probe lasers to the tissue surface and at specific diameter D. This assumes that the delivery optical elements for these two beams have their focal points not at the target tissue surface, especially for a large diameter D of the laser beams.

With further reference to FIG. 1, efficient optical detection of PNBs in a tissue surface layer within the tissue depth range h=0-200 um requires the focal point/plane of light collecting optical elements to coincide with PNBs, i.e. to be close to the tissue surface.

The laser device delivers laser beams to a tissue surface to generate one or more PNBs and to illuminate the one or more PNBs with a probe laser beam. The laser device may comprise any one or combination of the following features: (i) may use free space and fiber optical elements; (ii) the pump and probe beam can be co-delivered through the same or separate optical elements; (iii) optical delivery elements can be partially integrated with the light collection optical elements or be separate from it; (iv) the wavelengths of the pump and probe laser beams should be different enough to ensure a spectral separation of the two beams in the device; (v) the pump laser beam should be a picosecond pulse with the duration 5-40 ps and the adjustable fluence at the tissue surface in the range from 10 mJ/cm2 to 150 mJ/cm2; (vi) the spatial intensity profile of the pump laser beam at the tissue surface should be uniform and without “hot spots” i.e. without areas with the optical intensity being significantly higher or lower of its average value across the beam aperture at the tissue surface; (vii) one or several pump laser pulses should be delivered on demand to specific location of the tissue surface; (viii) the probe laser beam can be a continuous or pulsed radiation. In case of the probe laser pulse, its duration should be in the range 100 ps-20 ns, the probe pulse should be time-delayed relative to the pump laser pulse by specific time delay, in the range 10 ns-50 ns.

Collection of PNB-scattered probe laser light from the tissue surface may comprise any one or combination of the following features: (i) may use free space or fiber optical elements; (ii) collection optics may be partially integrated with the delivery optical elements; (iii) PNB-scattered light can be detected as an integrated time-response by using a photodetector or a time-resolved image by using an image detector; (iv) for detecting an integrated time-response, the collection optics may use aspheric lenses with high numerical aperture and should collect and deliver into a photodetector the maximal amount of PNB-scattered probe laser light; (v) for detecting a time-resolved image, the collection optics may use objective lenses with high numerical aperture and the field of view in the tissue surface having the size D of the laser beams or larger.

Optical detection of PNBs can use a single optical input photodetector to analyze the probe light scattered from the sample area irradiated by the pump laser pulse or it may use two optical inputs, one as described above, and the second one as an optical reference, from the tissue or other object which was not exposed to a pump laser pulse, and to process both optical inputs in the way that increases the PNB detection in the optical background of the tissue or of the intensity noise of the probe laser source or other background noise.

The present invention also provides for a tissue sample holder. In a preferred embodiment, the tissue sample holder design provides for quick and simple mounting and replacement of the samples and comprises one or more of the following features (i) position of the tissue surface (the position along optical axis of pump laser beam) should be stable, relative to the position of the pump and probe laser beams, during the replacement of the samples, with the reproducibility error+/−20 um or less; (ii) at least one optically transparent side between the tissue and the laser beams; (iii) the sample surface being scanned across the laser beams, in sync with the generation of pump laser pulses, so to continuously expose the desired area of the sample.

A pump and probe laser beams may be directed to specific locations of the sample or can be scanned across the desired area of the sample, identically exposing various locations to the pump and probe laser energies. This can be achieved by moving (scanning) the sample or the laser beams or both

Preparing a tissue sample for analysis may comprise the following steps: (i) identification and marking of surgical margins, if any; (ii) incubation of the sample for 10-30 minutes under physiological conditions, or under other specific conditions, with a plasmonic nanoparticle (PNP) conjugated to a monoclonal antibody (PNP-MAB) as soon as the sample is obtained, in which time PNPs will bind to and cluster around target cells; (iii) wash off unbound PNPs; (iv) place the sample to the sample holder, with the surface to be probed in contact with the holder side facing laser beams; (v) add secure the sample so it does not change its position uncontrollably.

In some embodiments the PNP-MAB conjugates comprise 200 nm hollow spherical shells for 1064 nm, or 60-150 nm spherical shells, hollow or silica core, for 532 nm, or solid metal spheres made of gold or another metal, for other pump laser wavelengths, which can be in a near-infrared spectral range between 700 nm and 800 nm.

The PNP-MAB conjugates comprise spherical shells of 60-200 nm in size. The spherical shells may be hollow or comprise a silica core. In an embodiment, the PNP-MAB comprise 200 nm hollow spheres for 1064 nm wavelength applications. In another embodiment, the PNP-MAB comprise 60-150 nm hollow spheres, for 532 nm applications. In another embodiment, the PNP-MAB comprise 60-150 nm hollow particles, for applications involving the pump laser wavelengths, which can be in a near-infrared spectral range between 700 nm and 800 nm. The PNP may be comprised of any plasmonic material, for example metals or metal-like materials. In a preferred embodiment, the PNP comprises a gold nanoparticle. The MAB may comprise any monoclonal antibody that targets the PNP to a particular cell type. By way of example but not limitation, in a preferred embodiment the MAB is Panitumumab, an antibody specific to the epidermal growth factor receptor (EGFR), which is often up-regulated in cancer cells.

To acquire a signal from the sample, the sample at the holder is placed on the laser device, the laser device comprising a free space pump laser beam and free space probe laser beam.

The free space pump laser beam comprises the following features comprises the following features 1064 nm or 532 nm wavelength, 5-40 ps pulse duration, 30-100 uJ pulse energy, 0-100 Hz, or 0-1000 Hz pulse repetition rate, pulse quality that is round with no hot spots, preferably flat top intensity distribution, 1-2 mm beam diameter, and external or internal single pulse triggering with an output electrical sync signal, wherein the features are optimized to maximize the signal ratio between diseased tissue and normal tissue. By way of limitation but not example, one commercially available laser that meets these features is the IPG Photonics YLPP fiber picosecond laser.

The free space probe beam comprises the following features: 600-700 nm or a 1300-1600 nm wavelength, 0.5-10 mW, a relative intensity noise within of 0.1% peak to peak in the frequency range of 0.01-100 MHz, a beam quality close to a round Gaussian beam, delivered collinearly with the pump laser beam, and illuminates the same field as the one exposed to the pump laser beam.

Both the free space pump beam and free space probe beam are delivered through a hole in a 45 diagonal mirror and high NA (0.4 or higher) lens, allowing for slight focus of these two beams but still allowing the beams divergent. The PNB-backscattered probe light is collected and collimated by that lens, reflected by 45 diagonal mirror, and focused by the second lens onto the photodetector. The photodetector should have a low noise in the bandwidth of PNB signal, for example within 0.1% peak-to-peak amplitude of the base level amplitude, in the bandwidth 0-100 MHz or other frequency bandwidth matching the bandwidth of PNB signal. A pinhole as a spatial filter may be used to improve the sensitivity. The sample is mounted into a sample holder, with its diagnostic surface towards the laser beams, and secured to prevent its unintentional movement in the holder. The sample holder is scanned in one or two lateral coordinates to expose the whole surface of the sample to the pump laser beam. The scan speed is determined by the pump pulse repetition rate (for example, 100 Hz×0.25 mm² (beam footprint area)=25 mm²/s).

In another embodiment, the picosecond pump laser pulse energy is delivered to the sample through an optical fiber selected from the group: (i) single multi-mode step index fused silica core fiber with the core diameter from 100 um to 400 um; (ii) hollow core fiber with a core diameter of 20 um to 200 um; (iii) or a bundle of several fibers mentioned above. In some embodiments, a bend radius of the fiber or a fiber bundle should be within 15 mm or less.

The optical fiber delivers single laser pulses out of its distal tip. In a preferred embodiment, the optical fiber delivers single laser pulses of PNB-specific duration/minimal fluence combinations at the wavelength 1030-1070 nm, or 532-800 nm. By way of example but not limitation, the pulse duration and fluence are as 10 ps and 50 mJ/cm2, respectively, (the pulse energy and average power for 200 um core and 1 KHz: 15 uJ/15 mW); 30 ps and 150 mJ/cm2 (45 uJ/45 mW); or 60 ps and 300 mJ/cm2 (90 uJ/90 mW).

For the pulse repetition rate up to 1 KHz and 200 um core fiber, these requirements result in an average power in mW range so no thermal damage to the optical fiber or its connectors should be expected. However, the surface and bulk damage to the optical fiber caused by optical breakdown and other non-linear optical effects of the laser pulse should be avoided. In case of fused silica core fibers, the bulk damage intensity threshold is close to 400 GW/cm2 (although for continuous laser the laser damage threshold for optical fibers decreases for 5 Gw/cm2). A preferred solution is in using optical fibers with non-resonant hollow core optical waveguide. Such fiber can deliver a pump laser pulse or both pump and probe laser beams.

In another embodiment, optical fiber or several optical fibers may be used to collect the light of a probe laser which has been scattered by PNB in the sample. Such fibers may be in a direct optical contact with a sample holder or use an interface optical element to collect the PNB-scattered probe laser light from PNBs in the sample and into the collection optical fibers

In another embodiment, a sample holder with the sample is not being scanned. Instead, an optical element is canned across the sample holder. Such optical element delivers the pump and probe laser beams to the sample and may also collect the PNB-scattered probe laser light from the sample. Such optical element can be connected to optical fiber or fibers which deliver the pump and probe laser beams from the laser sources to the element and the PNB-scattered probe laser light from the element to the photodetector.

To analyze the signal acquired from the sample, a time-response is registered with a photodetector and then converted into an electrical output signal of the photodetector. This electric signal includes: the baseline probe laser intensity signal, permanent for each location, generated by the backscattering from the tissue; and, if PNBs are present, a PNB signal that will change the scattered light baseline as a PNB-specific time-response. Parameters of such time-response, analyzed against diagnostic thresholds, will indicate the presence of cancer cells. The X-Y map for the sample slide can be analyzed as a PNB image. Optionally, the scan area can be optimized before the scan by obtaining the boundaries of the sample.

To detect and analyze the signal from the sample, the following algorithms can be used: insert PNB algorithms here

The present invention also provides for a system for detecting PNBs ex vivo, the system comprising two lasers, ps pulsed pump and c.w. probe, free space or fiber optical setup for delivery to and collection from the sample of the laser radiation of the pump and probe lasers, one or several photodetectors with signal digitizer(s), sample holder with the scanner in lateral directions, a computer-based program to control the pump and probe laser, the scanner, the digitizer, a photodetector, optionally a camera (to generate a sample boundary image) and the software. Optionally, the system may further comprise an incubation chamber and PNPs to prepare the sample for the laser exposure. To ensure high sensitivity of PNB detection, the probe laser and photodetector comprise a low amplitude or intensity noise. The laser footprint will be optimized for the maximal cancer (or other target) cell detection sensitivity.

To ensure diagnostic sensitivity in detecting PNB-generating target cells, the diameter of the probe laser will be optimized. The smaller is a probe laser beam diameter, the lower is an optical background and the higher is the PNb detection sensitivity. The larger is the probe beam diameter the higher is the probability of detecting a PNB in a target cells, but the background signal also increases with the probe beam diameter. For example, the optical scattering cross-section of a PNB with a diameter Dpnb up to 1 um can be estimated as ¾Dpnb² and would be close to 1 um². The optical cross-section of diagnostic events will be proportional to the number N of the generated PNBs, which can be approximated by the number of cancer cells: ¾Dpnb²×Num². The cross-section of the background signal can be estimated by the area of the footprint of the probe beam, ¾D²−¾Dpnb²×N. In case D>20 um the background optical scattering cross-section can be approximated by ¾D².

For the integral time-response, the background scattering determines the baseline signal Sb,

$$Sb = Kb 3 / 4 {D}^{2}$$

and the PNB scattering determines a diagnostic signal Sd,

$$Sd = Kpnb N 3 / 4 {Dpnb}^{2}$$

where Kb and Kpnb are optical scattering coefficients for the tissue and for PNB, respectively. Generally, Kpnb>>Kb, for example Kpnb=10Kb, and the maximal diameter Dpnb of the PNB is determined by the fluence of the pump laser pulse Fp as Dpnb A Fp, where A is a constant determined by the nanoparticle properties and by the nanoparticle binding to cancer cells. Therefore, the ratio Sd/Sb of the diagnostic signal to background signal, for integral time-response, would be:

$$Sd / Sb = [ Kpnb / Kb ] [ {Dpnb}^{2} / {D}^{2} ] N$$

This parameter should exceed the amplitude noise of the baseline by at least factor of 2, in order to detect the number (“N”) of target cells. For an ultimate detection of a single cancer cell (N=1) and assuming Kpnb/Kb=10, the typical noise level 0.01, D=20 um, Dpnb=1 um, the ratio above would be 0.025, vs the noise 0.01, and thus it will be possible to detect a single cancer cell in a sample. However, the diameter of the probe beam in this case is close to the size of a cancer cell.

While increasing the diameters of the pump and probe laser beams to a larger surface of the sample would allow for increasing the signal by the factor of N, the background signal will increase proportionally to the square of the diameter of the probe beam, and the ratio of the diagnostic signal to the background would decrease. The present invention provides for increasing the probe beam diameter using any one or more of the following features: (i) increasing optical scattering by a single PNB through the higher fluence of the pump laser pulse (by the factor of 2-5); (ii) increasing the ratio Kpnb/Kb (for example, from 10 to 100, i.e. by the factor of 10); (iii) reducing the amplitude noise of the probe laser (by the factor of 10, from 0.01 to 0.001 or to 0.1%). These steps potentially increase the ratio of Sd/Sb up by additional factor of 500 (5×10×10) and thus will compensate a similar increase in the probe beam diameter by 20 times (a 400-fold decrease in signal to background ratio). This would make a probe beam diameter of 200 um quite realistic to detect a single cancer cell through a PNB in the background of tissue-associated optical scattering. Therefore, small probe laser beams provide the highest diagnostic sensitivity and the lowest level of the energy of pump laser pulse.

Increasing the diameter of the probe laser beam increases the sample area probed by the method, providing that the diameter of the pump beam is increased proportionally. For the sample of finite size being scanned by the laser beams larger beam diameters reduce the time to result. However, this time depends also upon the repetition rate. With 1 MHz rates available, it may be easier to scan the sample fast enough with a small beam. For example, 20 um probe beam scan rate would be 3 cm²/s (at the linear scan speed of 20 m/s), sufficient for a real-time diagnostics of surgical and biopsy samples. However, 1 MHz digitation rate for a signal may not be available. If signal digitation is limited to 1 KHz rates, the scan speed would be, for example, 0.003 cm²/s (7 min for 3 cm² at 1 KHz scan, scan speed 20 mm/s) for a 20 um beam, or 0.3 cm²/s (10 s for 3 cm2, at 1 KHz scan, scan speed 200 mm/s) for a 200 um beam.

In an embodiment, the signal parameters obtained from the time-response during the scan can be mapped as X-Y image or multiple images, one image for each signal parameter, creating a digital diagnostic scanning PNB microscope.

In another embodiment, PNBs can be detected as the time-resolved scattering image with the pulsed probe laser. This solution allows to use larger diameter of pump and probe beams, and the Sd/Sb ratio will be determined by different factors, for example, the background will be determined by optical scattering non-uniformity of the tissue and the signal will be determined by the optical scattering brightness of PNB which is determined by maximal diameter of PNB which, in turn, depends upon the number of clustered around cancer cells plasmonic nanoparticles and by the fluence of the laser pulse. In this embodiment, a fast image sensor and analyzer is required that has the ability to capture, feed and analyze more than 1000 frames per second, and a higher energy of the pump laser pulse.

The present invention also provides for a motorized scanning stage for a sample holder, with a travel range of 50×50 mm, travel speed greater than 80 mm/s in at least two coordinates, travel accuracy of 10 um or better, known and encoded position, software-controlled. The stage should move the sample relative to the laser beams at the speed up to 200 mm/s in two coordinates, under computer program control. Examples of commercially available scanning stages that support travel speeds from 85 to 250 mm/s include Zaber and Thorlabs.

In an embodiment, to generate cancer associated PNBs at a tissue surface, single laser pulses of the duration/minimal fluence combinations are used for the laser beam diameter 50-200 um. For a 200 um diameter of the pump laser beam at the tissue surface the other parameters would depend upon pulse length:

Minimal pump pulse energy, uJ/average power,
mW, for the maximal repetition rate of 1 kHz

| | | | |
|---|---|---|---|
| Pump pulse duration, ps | 10 | 30 | 60 |
| Minimal pump pulse fluence, mJ/cm2 | 50 | 150 | 300 |
| Small tissue area, 20 um spot | 0.15/0.15 | 0.45/0.45 | 0.9/0.9 |
| (microscopy mode) | | | |
| Large tissue area, 200 um spot | 15/15 | 45/45 | 90/90 |
| (scan mode) | | | |

For the pulse repetition rate up to 1 KHz, these requirements result in an average power of the pump laser in mW range. There are multiple picosecond laser available with the pulse duration from 5 to 40 ps, the wavelength 532 nm, or 700-800 nm or 1030-1064 nm, with the pulse repetition rate from 0 to 1 KHz.

The present invention provides for a method of diagnosing disease, the method comprising the following steps:

Step 1. Incubating a Tissue Sample with Plasmonic Nanoparticles.

An ex vivo tissue sample, such as biopsy or surgical specimen, is incubated with specific plasmonic nanoparticles. Plasmonic nanoparticles may have specific shape, size, concentration and molecules attached to target and bind to cancer cells at and near the surface of the sample. Important is a short incubation time, 5-45 min. During the incubation, plasmonic nanoparticles will bind to and cluster around or in target cancer cells at and near the sample surface. After the incubation, unbound nanoparticles will be washed off from the sample.

The tissue sample may be alive, solid, soft or liquid, one piece or dissected into many components (e.g., for analyzing additional specific surfaces or the volume of the sample).

The tissue sample may be incubated with nanoparticles under physiological conditions of 5% CO2 and 37 C, to stimulate clustering of plasmonic nanoparticles through the mechanism of receptor-mediated endocytosis. In another embodiment, incubation at low temperature, 4C, to accumulate more plasmonic nanoparticles at the cellular membrane.

Step 2. Mounting the Sample onto an Optically-Transparent Holder.

The incubated tissue sample, or its specific surface, or its part, is mounted in an optically transparent holder, to form a flat wet tissue surface in contact with optically transparent surface for pump and probe laser beams

Tissue is covered from an opposite side with another glass or other rigid cover and is pressed against the holder surface to reduce the thickness of the sample, for example, to 0.2 mm so to allow the pump laser energy through the whole depth of the tissue

The holder can be scanned across the laser beam with the speed synced with the pump pulse repetition rate so to expose different locations of the sample to pump laser pulses.

In some embodiments, the sample holder may have two parallel glasses with a thin gap for a sample so the probe light scattered by PNB efficiently travels forward, for example, two microscope slide glasses.

In some embodiments, the sample holder may have an additional prism below the parallel glass so to support a total internal reflection of probe laser beam by a pump pulse-induced plasmonic nanobubble (which is a vapor) at the sample-glass border.

In some embodiments, the sample holder may be scanned across laser beams at specific speed so to expose additional locations of the sample to next pump laser pulses, and thus to expose the whole sample to the pump laser pulses for diagnosis the whole area of the sample surface.

In some embodiments, the sample holder may include a flow cuvette with a flowing sample.

In some embodiments, the sample holder may be modified to be used with optical or photothermal microscope, by using a microscope slide or other microscope-compatible transparent glass.

Step 3. Generating Plasmonic Nanobubbles with a Pump Laser Pulse.

The sample surface is exposed to a pump laser beam that delivers a pump laser pulse to the sample surface. The diameter of the laser beam at the sample is from 20 um to 200 um. The duration, fluence and wavelength of the pump pulsed beam are tuned to generate plasmonic nanobubbles (PNB) if that beam interacts with clusters of plasmonic nanoparticles at the surface of the sample.

In some embodiments, the pump laser pulse is focused to a spot of the diameter 10-100 um, for example, 30 um, at the sample surface. In some embodiments, the pump beam is focused into the surface of the sample.

In some embodiments, the focal point of the pump laser beam is scanned along the beam axis so to create the PNB generation conditions at specific depth range of the sample, for example, from 0 to 50 um, from 50 um to 100 um, from 100 um to 150 um deep.

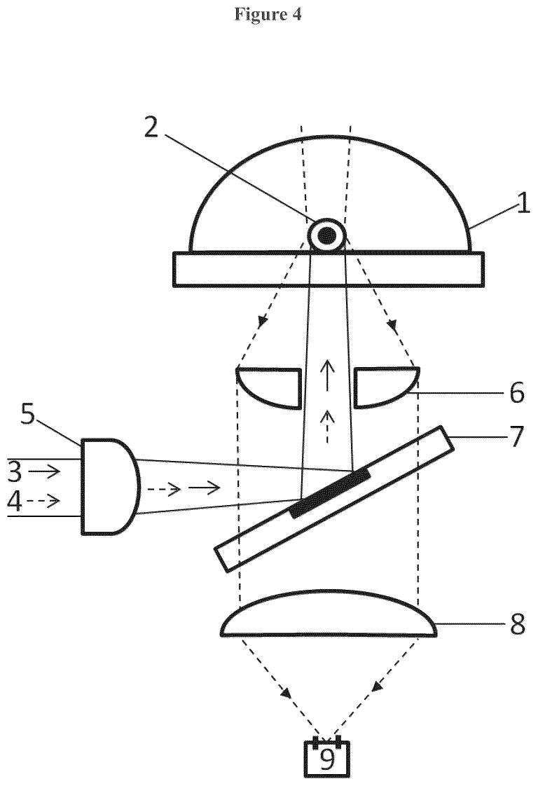

In some embodiments, the pump beam is focused or concentrated to a specific diameter on the sample with a lens which controls the diameter of the pump laser beam at the sample surface, for example, with an aspheric lens with low NA or with a collimator lens (FIG. 3).

In some embodiments, the pump laser beam is directed into the sample surface with a combination of the hole in the probe beam collecting mirror and a hole (shaft) in a probe beam collecting lens (FIG. 3).

In some embodiments, the pump beam is directed into the sample surface with a combination of three key elements (FIG. 4): the beam-forming lens (5), a small mirror (7) with its diameter close to the diameter of the laser beams at the mirror, and a hole (shaft) in a collecting lens (FIG. 4). The desired diameter D of the pump laser beam at the tissue surface is achieved through a combination of the distance of the lens (5) to the tissue surface and the focal length of the lens (5).

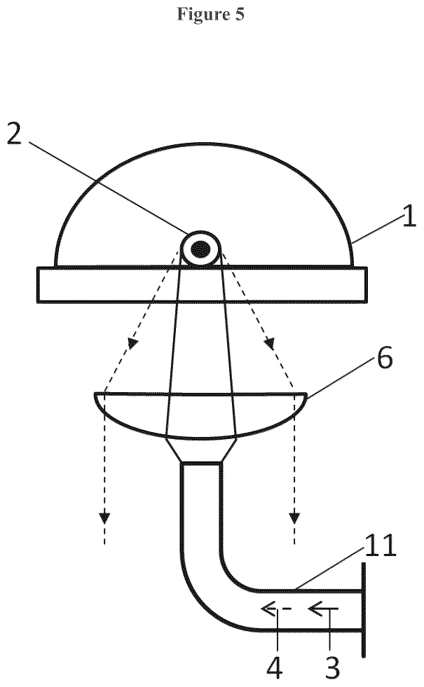

In some embodiments, the pump laser beam is delivered and concentrated to a specific diameter on the sample surface with the combination of two elements (FIG. 5): an optical fiber (11) and a collecting lens (6), optionally aspheric high NA lens, which also controls the diameter of the pump laser beam at the sample surface (FIG. 4). The desired diameter D of the pump laser beam at the tissue surface is achieved through a combination of the distance between the fiber tip and the back surface of the lens.

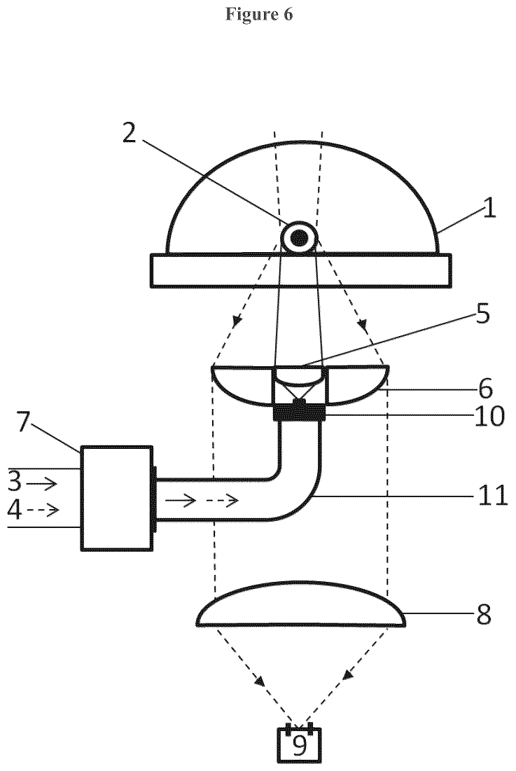

In some embodiments, the pump laser beam is delivered and concentrated to a specific diameter D on the sample with a combination of an optical fiber, with the core diameter from 50 to 400 um (10) and a fiber collimating (focusing) lens (5), which optionally is an aspheric or a grin lens, which controls the diameter of the pump laser beam at the sample surface (FIG. 6), for specific focal length and working distance of the collecting lens (6).

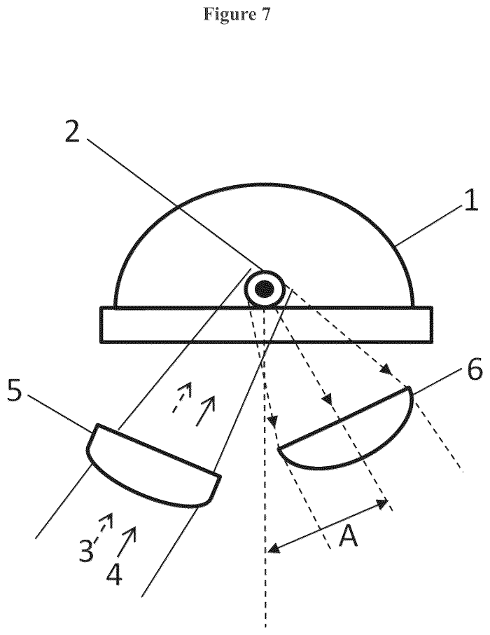

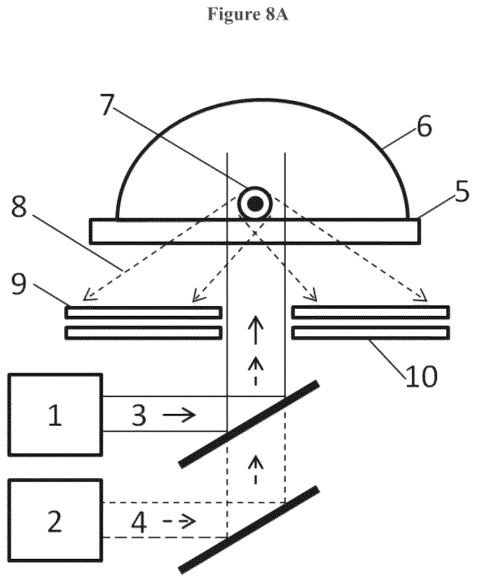

In some embodiments, a pump beam is delivered to the sample by using separate optic elements which do not collect a PNB-scattered light. The pump beam, in this case, can be delivered at some angle, so to allow sufficient space for a separate light-collection optics (FIG. 7). In this case of a separate delivery and collection, the delivery optical element, such as a lens (5), its parameters and position, can be optimized to directly project the beams onto a tissue surface, and the collecting optical element (6) can be independently optimized to maximize the collection of light from PNB generation zone (2).

In some embodiments, use two simultaneous laser pulses of two different wavelength both being absorbed by plasmonic nanoparticles, to improve the PNB generation and hence the diagnostic sensitivity.

In some embodiments, use a single laser pulse with the fluence above specific threshold associated with the generation of plasmonic nanobubbles in cancer cells, for example, 50 mJ/cm² for 30 ps laser pulse.

In some embodiments, a pump pulse has a specific duration in a picosecond time range, from 1 to 40 ps, to generate PNBs by using non-stationary pump laser pulse-induced transient plasmonic properties of metal nanoparticles, for example, at 782 nm or 1064 nm although stationary plasmonic optical absorption peak of such nanoparticles can be quite different, for example, in the range 540-650 nm.

In some embodiments, a single pump laser pulse is applied to each location of the sample surface which has not been previously exposed to a pump laser, and many adjacent locations are probed by scanning the sample surface across the pump laser beam.

In some embodiments, several identical pump laser pulses are applied to the same location of a tissue surface, to analyze the difference in the response signals associated with each of such pump laser pulses.

In some embodiments, several different pump laser pulses are applied sequentially to the same location: two pump laser pulses at different wavelength and with time delay of 0.1-10 us, to generate PNBs around two different types of plasmonic nanoparticles associated with two different types of target cells or with two different molecular targets representing different cancer types, stages or other properties of diagnostic interest. In this case, each laser pulse may produce a separate time-response or image signals to show PNBs associated with two different targets of disease types.

### Step 4. Exposing the Plasmonic Nanobubbles to a Probe Laser Beam.

The sample surface is exposed to specifically aligned probe laser beam of the diameter (at the sample) in the range from 10 um to 200 um and that delivers a probe laser to the same location of the sample surface as the one exposed to the pump laser beam. The pump laser pulse-generated plasmonic nanobubbles are detected optically through the scattering of probe laser beam (8) by plasmonic nanobubbles.

In some embodiments, the wavelength of probe laser beam is significantly different from that of the pump laser pulse.

In some embodiments, the probe laser beam is continuous and is co-delivered with a pump laser beam so their apertures are close or coincide at the sample tissue surface, and its power is limited to 5 mW.

In some embodiments, the probe laser beam is a single pulse, with the duration from 100 ps to 20 ns, and it is delayed relatively to the pump laser pulse by the delay of 10 ns or other specific delay in the range from 10 ns to 50 ns, and is co-delivered with the pump laser pulse so their apertures are close or coincide at the sample tissue surface.

In some embodiments, the probe beam uniformly illuminates the tissue surface within the aperture D of the pump laser beam at the tissue surface.

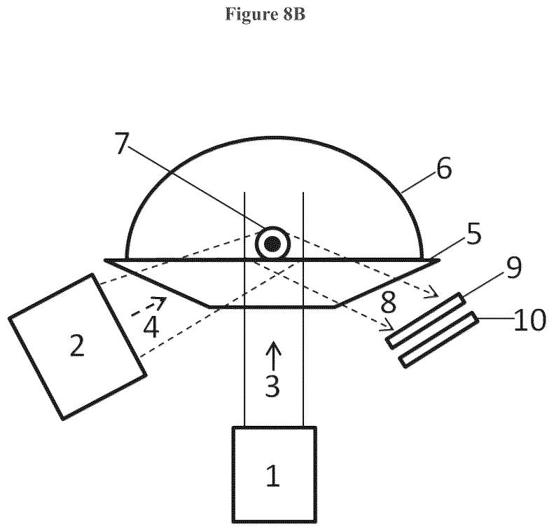

In some embodiments, a probe laser beam is directed into a tissue separately from a pump laser beam (FIG. 8B), with a separate optical element, at the angle which is below the critical angle of internal total reflection for liquid or tissue but above the critical angle of internal total reflection for a vapor: in this case, a PNB would create a total internal reflection of the probe beam from the sample surface, and position the photodetector to specifically detect probe light caused by a total internal reflection.

In some embodiments, two or more probe laser pulses are applied to each tissue location: the 1^{st} one before the pump laser pulse, and the 2^{nd} one and next probe laser pulses applied to the same location, each within specific time-delay relative to the pump laser pulse, in the range from 10 ns to 50 ns.

### Step 5. Diagnosing the Sample Based on the Signal of the Probe Laser Light.

A probe laser light is detected with an optics (including a filter (9)) and a photodetector (10), FIG. 8) positioned to collect sufficient amount of backscattered by a plasmonic nanobubble probe laser light. A plasmonic nanobubble-specific signal out of photodetector would report a cancer cell in the sample.

In some embodiments, a PNB-backscattered light is collected with high-NA lens (a probe beam collecting lens) mounted in front of the holder, with the focal point at the PNB generation site (FIG. 3-7): such lens collects the maximal amount of the PNB-backscattered light and collimates and directs that light into a photodetector, through an additional lens and optical filter.

In some embodiments, a probe beam collecting lens has a hole (a bore shaft) (FIG. 4) to pass the pump and probe beams to the sample through that hole (shaft), while the pump and probe beams are controlled by a separate lens.

In some embodiments, a probe beam collecting lens has an optical fiber connector and a fiber collimating lens installed into its hole (shaft) (FIG. 6), with an optical fiber that delivers the pump and probe beams to the sample.

In some embodiments, a probe beam collecting lens is an aspheric lens or a ball lens with anti-reflective coating at the wavelength of the probe beam and with high numerical aperture, in the range 0.5-1.0, and a lens or a system of lenses (6) and (8) (FIG. 6) efficiently couple the PNB-scattered light, from the zone exposed to a probe laser beam in the tissue surface, into a photodetector, to form an integrated time-response with continuous probe laser light, such time-response showing a PNB-specific signal(s) in case PNB(s) is/are generated within the tissue surface zone exposed to a probe laser beam.

In some embodiments, a probe light collecting lens is mounted at some angle (FIG. 7) to collect PNB side-scattered continuous probe light and to direct it to a photodetector.

In some embodiments, a probe light collecting lens (6) transmits the image of a sample surface area, the one exposed to a probe laser pulse, into an image detector. A lens (6) or a combination of lenses (6) and (8) form an image of the tissue surface area at the image detector and so image PNB(s) generated in the tissue surface and within the aperture of a probe laser, with the pulsed probe laser light which is delayed relative to the ump laser pulse.

In some embodiments, continuous probe light is detected, and the integrated optical signal is analyzed as a time-response, in the time window from 0 to 1 us after a pump laser pulse. The time window length is, in this case, 1 us, and it can vary from 0.5 us to 50 us, in response to each pump laser pulse.

In some embodiments, multiple time responses are detected, each for specific location of the sample during its scan, and generate an image (a map) of time-response parameters for the scanned area of the sample.

In some embodiments, at least two different probe laser beams are compared and processed optically or electrically to subtract the noise or background in the way that increases the sensitivity of PNB detection, for example, by using balanced photodetectors with two optical inputs, and registering the probe beam light delivered from two zones, one exposed to a pump laser pulse and the other, reference zone, not exposed to a pump laser pulse

In some embodiments, a time-response signal is processed to determine and quantify PNB-related parameters of the signal.

In some embodiments, a time-response signal is processed with specific frequency filters to improve the PNB-specific components of the signal. The frequencies for high-pass and low-pass filters can be derived from the range of PNB lifetimes, for example, in the range from 10 ns to 400 ns

In some embodiments, multiple time responses are detected, each for specific different location X,Y of the sample surface during its scan, and generate an image (an X,Y map) of time-response signal parameters for the scanned area of the sample.

In some embodiments, more than one time-response signal is detected from the same location in response to more than one identical pump laser pulses to the same location and compare signals after the 1^{st} and next pump laser pulses. Analyze the signal decay and use specific threshold to diagnose cancer.

In some embodiments, the collected probe laser light is detected as an image, with an image detector, so to create an optical scattering image of the sample surface. Identify PNBs in the detected image as parts of the detected image with the pixel amplitude above specific threshold

In some embodiments, a pulsed probe light is detected as two images for the same location, before and after the pump pulse, and analyze the relative change of a pixel intensity in each location to identify PNBs.

In some embodiments, cancer is diagnosed by comparing the signal, a time-response or an image, parameters to specific thresholds.

In some embodiments, an automated algorithm is used to diagnose cancer through analysis of signal parameters, and generate the diagnosis in a qualitative or quantitative form for each sample or each location of the sample.

In some embodiments, an automated diagnosis is used to automatically guide and control a diagnostic or surgical robotic device or procedure, so to expedite the delivery of the treatment to the location diagnosed with cancer.

In some embodiments, more than one cancer is differentiated by detecting and analyzing the signals from different nanoparticles which targeted two different cancers, and were exposed to two different pump laser pulses tuned to selectively generate PNBs in one type of nanoparticles.

The foregoing method requires a combination of all 5 steps (components) to rapidly detect cancer cells in the sample with a high sensitivity, up to a single cell, and thus provides the detection of cancer in a biopsy or a surgical specimen within 20-30 min from a sample extraction from a patient. This short time to result, in turn, would support intraoperative guidance of the surgery or a treatment within a biopsy sampling procedure, to be delivered to a cancer-positive site within the primary procedure.

What is claimed is:

1. A method comprising (a) incubating a sample with plasmonic nanoparticles, (b) mounting the sample onto a holder, (c) exposing the sample to a pump laser, (d) exposing the sample to a probe laser, and (e) diagnosing the sample based upon the signal of the probe laser beam.

2. The method of claim 1, wherein the plasmonic nanoparticles comprise metal nanoparticles.

3. The method of claim 1, wherein the plasmonic nanoparticles comprise gold nanoparticles of about 50-150 um diameter.

4. The method of claim 1, wherein the plasmonic nanoparticles comprise a spherical surface.

5. The method of claim 1, wherein the plasmonic nanoparticles comprise hollow shells.

6. The method of claim 1, wherein the plasmonic nanoparticles comprise a monoclonal antibody.

7. The method of claim 1, wherein the plasmonic nanoparticles comprise at least two types of nanoparticles for excitation with a different optical wavelength.

8. The method of claim 1, wherein the holder is selected from: one flat glass, two flat parallel glasses, a flat glass and prism, and a cuvette.

9. The method of claim 1, wherein the step of diagnosing the sample comprises analysis of a light detected by a photodetector, wherein analysis is selected from one or more of detected time-responses of a c.w. probe beam, detecting a pulse (non-stationary component) from the probe laser, subtracting stationary background of probe light.

10. The method of claim 1, wherein the step of diagnosing the sample comprises comparison of the probe laser output signals before and within a specific time window after exposure to the single pump laser pulse.

11. A system for diagnosing a sample ex vivo comprising one or more plasmonic nanoparticles, a sample holder, a pump laser, a probe laser, a photodetector for capturing probe laser light from the sample, a computer, and a software module on the computer for comparing the probe laser light before and during exposure to a single pulse by the pump laser.

12. The system of claim 11, wherein the plasmonic nanoparticles comprise metal nanoparticles.

13. The system of claim 11, wherein the plasmonic nanoparticles comprise gold nanoparticles of about 50-150 um diameter.

14. The system of claim 11, wherein the plasmonic nanoparticles comprise a spherical surface.

15. The system of claim 11, wherein the plasmonic nanoparticles comprise hollow shells.

16. The system of claim 11, wherein the plasmonic nanoparticles comprise a monoclonal antibody.

17. The system of claim 11, wherein the plasmonic nanoparticles comprise at least two types of nanoparticles for excitation with a different optical wavelength.

18. The system of claim 11, wherein the holder is selected from: one flat glass, two flat parallel glasses, a flat glass and prism, and a cuvette.
